(19) United States
(12) Patent Application Publication
(10) Pub. No.: US 2004/0127915 A1
(43) Pub. Date: Jul. 1, 2004

(54) Suture hoop system

(76) Inventors: Richard P. Fleenor, Englewood, CO (US); Robert L. Bromley, Littleton, CO (US)

Correspondence Address: Russell T. Manning, Esq., MARSH FISCHMANN & BREYFOGLE LLP, Suite 411, 3151 South Vaughn Way, Aurora, CO, 80014, US

(21) Appl. No.: 10/331,520

(22) Filed: Dec. 30, 2002

(51) Int. Cl. A61B 17/04

(52) U.S. Cl. 606/144

(57) ABSTRACT

A suturing needle is provided that includes a selectively extendable grasping member disposed within an internal passageway for engaging suture material and holding that suture material relative to the needle during suturing procedures. In this regard, suture material may be grasped relative to a needle, inserted through tissue and released from the needle upon which the needle may be retracted leaving the suture material disposed through the patient tissue. In one embodiment the grasping member is disposable through a side aperture of the needle. In a further embodiment, the grasping member is operable to form a suture engaging loop relative to an outside surface of the needle. In another embodiment, the size of this suture engaging loop is adjustable to provide enhanced suturing capabilities.

### FIELD OF THE INVENTION

The present invention relates to the field of minimally-invasive internal surgery, and the apparatus and methodology for suturing within a patient body via the manipulation of devices external to a patient body. More particularly, the present invention is directed to a suturing needle that incorporates a grasping mechanism that is particularly apt for use in minimally invasive internal surgery.

### BACKGROUND OF THE INVENTION

Minimally invasive internal surgery procedures are ever increasing. Such procedures typically entail the insertion of one or a plurality of tubular members into a patient body and the passage of various devices through the tubular member(s) to access a tissue site of interest. Broadly, the term endoscopic device is used to describe a plurality of minimally invasive surgical devices or “scopes” that have been developed for specific uses. For example, upper and lower endoscopes are utilized for accessing the esophagus/stomach and colon, respectively, angioscopes are utilized for examining blood vessels, and laparoscopes are utilized for examining the peritoneal cavity.

In endoscope procedures, a single tubular member is typically inserted through a bodily orifice to provide instrumentation access therethrough to an internal tissue site, e.g., through the mouth or anus to access a hollow organ or other tissue lumen. Given such access points and associated bodily canals, the tubular members utilized in endoscopic applications are necessarily of flexible construction and may be of significant length. To date, endoscopic procedures have been largely limited to gastroesophageal and biopsy applications. However, it is believed that new surgical devices and procedures may be developed that will facilitate increased endoscopic applications involving the ligating, proximating and suturing of tissue in the stomach and colon.

Common to all endoscopic surgical procedures is the need to effectively suture an internal tissue site of interest. Such suturing entails the passage of a suture material into and back out of patient tissue at least once, and most typically a plurality of times, followed by the tightening of a knot incorporated with the suture material proximate to the sutured tissue. As may be appreciated, the completion of suturing procedures in endoscopic applications can present a challenging and sometimes tedious task for surgical personnel. For example, such suturing procedures may involve difficult manipulation of an external device to cause an internally located needle to pass entirely through tissue at a surgical site to effect suture stitching. Further, in some approaches, the provision of a suture knot at a sutured site may require the time-consuming removal and reinsertion of the entire suturing apparatus and access tubing therefore in order to tie a knot extracorporeally. In any case, surgical personnel require an effective means to grip and release suture material relative to an endoscopically controlled needle assembly to manipulate the suture material through a tissue site of interest.

### SUMMARY OF THE INVENTION

An objective of the present invention is to provide a suture needle that facilitates grasping suture material relative to the needle for endoscopic suturing.

Another objective of the present invention is to provide a suture needle assembly that allows for simplified remote suturing.

Another objective of the present invention is to provide a suture needle that has enhanced suture grasping capabilities.

A further objective of the present invention is to provide a suture needle incorporating a grasping assembly having a reduced profile.

One or more of the above-noted objectives and additional advantages are realized by the present invention that provides a suturing apparatus that includes a grasping member for engaging suture material and holding that suture material relative to the needle assembly during suturing procedures. In this regard, suture material may be grasped relative to a needle, inserted through a proximal side of tissue, released from the grasping member on a distal side of tissue at which time the needle may be retracted leaving the suture material disposed through the patient tissue. As will be appreciated, the portion of suture material disposed on the distal side of the tissue may be reengaged by the grasping member for further tissue engagement (e.g., making additional sutures).

In one aspect of the invention, the suturing apparatus includes a needle that terminates at a distal end for piercing patient tissue. The needle includes an internal passageway that extends through at least a portion of the needle and an aperture that extends through the needle to the internal passageway along a side surface of the needle. Furthermore, the apparatus includes a grasping member that extends through at least a portion of the internal passageway. The grasping member is disposed to allow selective movement through the internal passageway. In this regard, the needle and grasping member permit selective relative movement therebetween. Upon such selective relative movement, a portion of the grasping member is positionable through the side aperture of the needle for selectively grasping and/or releasing suture material during a surgical procedure.

Use of the side aperture to provide access to the internal passageway may allow the distal end of the needle to be closed to form a piercing tip. That is, the internal passageway may not extend through the distal end of the needle. Accordingly, the side aperture will pass through the needle body to the internal passageway at a point spaced from the distal end of the needle. In certain embodiments, the side aperture may be formed through the needle at a position near the closed distal end of the needle allowing suture material to be held relative to the needle at a position near the piercing tip. In any case, the closed piercing tip may be smaller than, for example, a hypodermic type needle where an internal passageway extends through the end of the needle. Accordingly, the closed piercing tip may be of a finer gage to provide enhanced piercing capabilities.

For endoscopic applications, the needle may be elongated with a maximum cross-sectional width, or diameter, of about 0.10 inches. Additionally, for such applications the needle may preferably comprise a resilient material that permits a degree of flexural curvature along the length thereof. By way of example, the needle may comprise a material selected from a group consisting of nickel, titanium, nickel-titanium alloys, stainless, spring or surgical steel, or in some cases, flexible plastics.

The internal passageway may extend through the majority of the needle and will be sized to permit at least a portion of the grasping member to move there through. However, in one embodiment the internal passageway is formed to restrict the movement of at least a portion of the grasping member. For example, the internal passageway may have a reduced cross-sectional area in one section that prevents the movement of a portion of the grasping member (i.e., having a diameter greater than the reduced cross-sectional area) through that section of the passageway. In this regard, a portion of the grasping member may be restrained within the internal passageway allowing tensile and/or compressive forces to be applied through the grasping member.

The grasping member may comprise any configuration that allows the grasping member to be disposed within the internal passageway for selective deployment through the side aperture of the needle while providing a means to selectively grasp suture material during surgical procedures. In one embodiment, the grasping member comprises a pre-formed loop that is collapsed in a non-deployed state when positioned within the internal passageway of the needle. This pre-formed loop automatically opens to a non-collapsed deployed state upon being positioning through the side aperture of the needle. This pre-formed loop may be selectively positioned about an end portion of the suture material when in a deployed state, and retract the suture material into the internal passageway in a non-deployed state, thereby securing the suture material to the needle.

In another grasping member embodiment, the grasping member comprises a filament wherein at least a portion of the filament extends through the internal passageway. One end of this filament may be fixedly interconnected (i.e., anchored) to the needle to allow forces to be applied to the filament for, inter alia, pulling suture material through the side aperture. In one embodiment, a distal end of the filament is fixedly interconnected within the internal passageway between the side aperture and the distal end of the needle. In this configuration, movement of a proximal end of the filament relative to the needle disposes a portion of the filament through the side aperture. As will be appreciated, the filament disposed through the aperture along with a portion of the needle may collectively define a loop relative to the outside surface of the needle for grasping a suture. Furthermore, unlike a grasping member having a pre-formed loop portion that is deployable through the aperture to define a loop of a predetermined size, the loop defined by the filament is selectively adjustable. That is, by disposing additional filament through the aperture, a larger loop may be defined relative to the outside surface of the needle. This selective control of the loop size provides enhanced ability to engage suture material during endoscopic procedures. Utilization of a single filament to form the grasping member provides another benefit, the ability to produce a suture needle having a reduced cross-sectional profile. As will be appreciated, grasping members that utilize a pre-formed collapsible loop require two wire members to be disposed adjacent one another within a portion of the internal passageway of the needle. In contrast, a single filament grasping member may utilize a smaller internal passageway and thereby allow production of smaller needles.

The filament may comprise any material that provides the desired mechanical characteristics required for forming a loop. These materials include, without limitation, beryllium-copper, nickel-titanium alloys, stainless steel, spring steels, and surgical steels. Furthermore, composite fibers (e.g., super elastic fibers) may also be utilized to form the filament. In any case, the filament should be an elastic material that permits flexural curvature along its length.

By retracting the grasping member relative to the needle, the loop disposal relative to the outside surface of the needle is retracted back into the internal passageway. Accordingly, this retracts any suture material that is disposed through the loop into the internal passageway and secures this material to the needle such that the needle, along with the suture material may be disposed through patient tissue. When the loop is disposed outside the needle, a rotational movement may be applied to a proximal end of the grasping member to manipulate the loop disposed outside the needle. That is, a rotational movement may allow for arcuate movements of the loop and/or twisting of the loop relative to a longitudinal axis of the needle. As will be appreciated, this manipulation allows for directional movement between the loop and the needle and enhanced ability to engage suture material during surgical procedures.

In a further embodiment of the first aspect of the invention, the assembly includes suture material having a pre-tied knot disposed about a portion of the needle. Generally, the suture material will be disposed near the piercing end of the needle allowing a free end of the suture material to be selectively engaged by the grasping member during surgical procedures. A second anchor end of the suture material may be affixed to a proximal portion of the suturing assembly to provide an anchor for the suture material.

In a further embodiment, the apparatus also includes a pusher member disposed relative to the needle to permit selective relative movement therebetween. In this regard, upon selective relative movement, the pusher member is able to engage the suture material to position the pre-tied knot beyond the distal end of the needle. That is, after one or more sutures are placed through a patient's tissue, a pre-tied knot is pushed off the needle and positioned to hold the suture material passing through the patient tissue in place. In one embodiment, the pusher member is a hollow tubular member that is in a concentric-coaxial relationship with a portion of the outside surface of the needle.

According to a second aspect of the present invention, a suturing apparatus is provided that includes a needle having an internal passageway that extends through at least a portion thereof; a grasping member that extends through at least a portion of the internal, passageway wherein and at least a first portion of the grasping member is restricted from passing through the internal passageway. That is, the grasping member may be attached to the internal passageway, or, may contain a portion sized to prevent its passage through the internal passageway. The needle and a non-attached portion of the grasping member are disposed to permit selective relative movement therebetween allowing a second portion of the grasping member between the first and second ends to be positioned through an aperture on the needle for grasping a suture.

In order to restrict a first portion of the grasping member from passing through the internal passageway, the internal passageway may include a section that is reduced in at least one dimension (e.g., diameter). As will be appreciated, this reduction allows, for example, one end of the grasping member to be restrained while a tensile or compressive force is applied to the second end of the grasping member. In this regard, the grasping member may be tightened or compressed (e.g. bowed), depending on the force applied. Accordingly, the second portion of the grasping member is advanceable or retractable through an aperture on the needle for engaging a suture or for pulling an engaged portion of suture material into the internal passageway.

The aperture passing through the needle to the internal passageway may be formed in any position along the length of the needle. That is, the aperture may extend through an end portion of the needle, or a side portion. So long as one portion of the grasping member is prevented from passing through part or all of the internal passageway. In any case, the aperture is formed to permit a portion of the grasping member to be selectively disposed through the aperture to engage suture material outside the needle assembly.

In one embodiment, the grasping member comprises a single, continuous filament. Accordingly, one end may be fixedly interconnected to the needle to provide an anchor point for the grasping member. As will be appreciated, this provides a grasping member that may have increased tensile strength characteristics. Furthermore, the single filament provides a suture engaging loop that has a selectively adjustable size. For example, in limited space surgical procedures, a small loop may be disposed through the aperture for engaging suture material.

According to another aspect of the present invention, a method for utilizing a suture apparatus is provided. The method includes the step of advancing a grasping member through an aperture extending through a side surface of a needle to at least partially define a loop relative to the outside surface of the needle. Once the loop is disposed outside the needle, a portion of a suture material may be engaged through the loop and the grasping member may be retracted into the internal passageway along with the suture material, which is then secured relative to the needle. Once the suture material is secured to the needle, the method may further include the steps of advancing a portion of the needle through a patient tissue site, along with the suture material secured relative to the needle. Once disposed through the patient tissue, the suture material may be released from the grasping member, at which time the needle may be withdrawn from the tissue site while a portion of the suture material is left protruding from the patient tissue. Accordingly, the grasping member may re-engage this protruding portion of the suture material and additional sutures may be performed.

The steps of extending and retracting the grasping member relative to the needle may each further include applying an axial force to a proximal end of the grasping member to produce an axial movement of the grasping member relative to the needle. As will be appreciated, this allows positioning a portion of the grasping member through the needle aperture. The step of engaging and disengaging the suture material may also include rotating the needle and/or grasping member to engage a portion of the suture material through the grasping member loop. Alternatively, the step of engaging and disengaging may include rotating a distal end of the grasping member relative to the needle to produce a twisting movement of the loop relative to the needle thereby moving the loop from a generally coplanar orientation with the needle to a non-planar orientation.

In conjunction with this aspect of the invention, the suturing apparatus may include a suture material having a pre-tied knot disposed about the suture needle. The suture apparatus may further include an outer member selectively positioned relative to the needle. The outer member may be utilized to position the pre-tied knot beyond a distal end of the needle for selective advancement and tightening with the suture material disposed through the patient tissue. As will be appreciated, in order to secure the suture material, the free end of the suture material protruding from the patient tissue must be re-engaged by the grasping member, secured to the needle and the knot positioned over the suture material. As will be appreciated, the method may further utilize real time viewing of the suturing process to allow for the remote manipulation of the suturing material and/or grasping member during an endoscopic procedure.

Additional aspects and advantages of the present invention will become apparent to those skilled in the art upon consideration of the further description provided hereinbelow.

### BRIEF DESCRIPTION OF THE DRAWINGS

FIGS. 1A-1D show first, second and third embodiments of grasping members utilized with the suture needle of the present invention.

FIG. 2 shows a perspective view of a laproscopic device utilizing the suture needle as shown in FIG. 1A.

FIG. 3 shows a cross-sectional view of the laproscopic device in FIG. 2.

FIGS. 4A-4C show manipulation of the laproscopic device of FIG. 2 to deploy the grasping member from the needle.

FIG. 5A shows a perspective view of an endoscopic device utilizing the needle assembly of FIG. 1A.

FIGS. 5B-5F show individual perspective views of the concentric, coaxial components of the endoscopic device of FIG. 5A.

FIGS. 6A-6C show cross-sectional views of the handle portion of the endoscopic device of FIG. 5A wherein various handles are extended and retracted relative to one another.

FIGS. 7A-7B show cross-sectional view and end cross-sectional of the tip portion of the endoscopic device of FIG. 5A.

FIGS. 8A and 8B show utilization of various components of the endoscopic device of FIG. 5A to manipulate suture material.

FIGS. 9A-9I show utilization of the needle assembly of FIG. 1A to perform an endoscopic suturing procedure.

### DETAILED DESCRIPTION

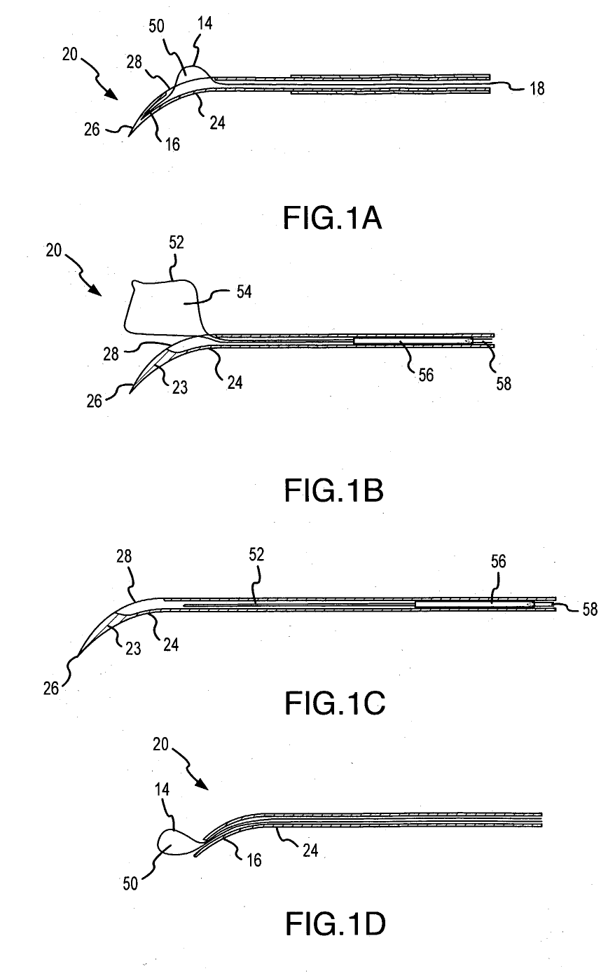

FIG. 1A shows a first embodiment of a suture needle assembly 20 incorporating a grasping member assembly that is particularly apt for use with minimally invasive surgical devices such as endoscopes, laparoscopes, and angioscopes.

As shown in FIG. 1A, the first embodiment of the needle assembly 20 includes a substantially hollow tubular member 24 that terminates on a distal end in a closed piercing tip 26. As shown, the needle assembly 20 has an arcuate configuration, however, it will be appreciated that the needle assembly 20 may utilize other configurations as well (e.g., straight). The needle assembly 20 further includes a slot opening 28 extending through a side surface of the hollow tubular member 24 which provides access to the internal needle cavity defined by the hollow tubular member 24. As shown, the slot opening 28 is located on the tubular member 24 near the piercing tip 26 to facilitate suture material manipulation, as will be discussed herein. However, it will be appreciated that the location of the slot opening 28 may vary.

Coaxially disposed within the hollow cavity of the tubular member 24 is a grasping assembly. In the embodiment of FIG. 1A, the grasping assembly includes a continuous wire 14 having a distal end 16 fixedly interconnected within the distal end of the hollow tubular member 24. More particularly, the distal end 16 of the wire 14 is affixed within the tubular member 24 at a point beyond the slot opening 28 and near the piercing tip 26 of the needle assembly 20. Any appropriate means may be utilized to interconnect the wire 14 to the tubular member 24, a non-inclusive list includes adhesively attaching the wire 14, crimping, welding and/or soldering the wire 14.

Except for the affixed distal end 16 of the wire 14, the grasping assembly and needle assembly 20 are able to move relative to one another allowing a lateral portion of the wire 14 to move between a deployed configuration and non-deployed configuration. In a non-deployed configuration, no portion of the wire 14 is disposed through the slot opening 28. That is, the wire 14 is seated within the hollow cavity of the tubular member 24 relative to the slot opening 28. In a deployed configuration, as shown, a lateral portion of the wire 14 extends through the slot opening 28 to define a loop 50 relative to a side surface of the needle assembly 20.

The size of the loop 50 may be adjusted by disposing additional wire 14 through the slot opening 28. That is, continued axial advancement of a proximal end 18 of the wire 14 toward its distal end 16 within the tubular member 24 results in a larger loop 50. Once extended to a desired size outside the tubular member 24, the loop 50 may be used to selectively engage suture material and hold that material relative to the needle assembly 20 during suturing procedures. That is, a portion of suture material may be disposed through the loop 50 while the wire 14 is in a deployed configuration whereupon the wire 14 may be retracted to the non-deployed configuration trapping the suture material between the wire 14 and the inside surface of the hollow tubular member 24. While in this trapped position, the suture material may be inserted through patient tissue along with the needle assembly 20, as will be more fully discussed herein.

To move between the deployed and non-deployed configurations, the wire 14 is moved axially relative to the hollow tubular member 24. For example, by advancing a proximal end 18 of the wire 14 relative to the hollow tubular member 24 (i.e., while the tubular assembly maintains a fixed position), a lateral portion of the grasping wire 14 is forced through the slot opening 28 of the hollow tubular member 24. That is, the wire 14 is compressed resulting in a portion of the wire flexing or bowing through the slot opening 28 and provides the suture engaging loop 50 on the outside surface of the needle assembly 20. By retracting the proximal end 18 of the wire 14, the lateral portion of the wire 14 is drawn back through slot opening 28.

FIGS. 1B and 1C show a second embodiment of the needle assembly that is substantially similar to the embodiment shown in FIG. 1A. As such, common reference numerals are utilized in the description provided above with respect to the commonly employed reference numerals as applicable. However, a number of alternative features in the embodiment of FIG. 1B should be noted. Particularly, the grasping assembly 10 of the embodiment of FIG. 1B utilizes a collapsible pre-formed hoop member 52 instead of the wire 14 utilized in the embodiment of FIG. 1A. Furthermore, the grasping assembly 10 is not fixedly interconnected to the tubular member 24.

The collapsible hoop member 52 is formed of a spring loaded wire-like material shaped to have an opening 54 therethrough. When the hoop member 52 is advanced through the slot opening 28, the hoop member 52 opens to a deployed configuration defining the opening 54. In order to be advanced through the slot opening 28, the tubular member 24 is filled between the piercing tip 26 and the slot opening 28. That is, an internal ramp 23 is utilized to direct the hoop member 52 through the slot opening 28. As will be appreciated, the hoop member 52 of this second embodiment is of a predetermined size. Further, the grasping assembly 10 also includes a connector 56 for connecting the ends of the hoop member 52 to a wire 58 disposed within the hollow tubular member 24. Accordingly, this wire 58 may be manipulated to advance and retract the collapsible hoop member 52 relative to the slot opening 28. Upon retraction into the hollow tubular member 24, the pre-formed hoop member 52 collapses to a non-deployed state. See FIG. 1C. As above, the hoop member 52 may be utilized to selectively engage and manipulate (i.e., grasp) suture material, as will be discussed herein.

FIG. 1D shows a third embodiment of the needle assembly that is substantially similar to the embodiment shown in FIG. 1A. Again, common reference numerals are utilized in the description provided above with respect to the commonly employed reference numerals as applicable. The embodiment of FIG. 1D utilizes a single wire 14 having its distal end 16 affixed within the internal bore of the tubular member 24. Again, the wire 14 is utilized to selectively define a suture grasping loop 50 outside the needle assembly 20. However, instead of utilizing the side slot 28 for disposing the loop 50 relative to a side surface of the needle assembly 20, the wire 14 passes through an aperture on the distal end of the tubular member 24. That is, the embodiment of FIG. 1D utilizes a hypodermic configuration where the loop 50 defined by the 142 is disposed in front of the needle assembly 20. In this regard, it will be noted that the wire 14 undergoes a 180° return bend upon being retracted within the tubular member 24.

Either of the above noted embodiments that utilize the side slot opening 28 for selectively deploying and retracting a grasping member 10 relative to the needle assembly 20 allow for the utilization of a closed piercing tip 26. As will be appreciated, this closed piercing tip 26 may be sharper than the piercing tip of an open ended needle assembly (e.g., hypodermic configuration of FIG. 1D) where a grasping member is advanced and retracted through a hollow end of the needle assembly. Furthermore, in the embodiment of FIG. 1A, utilization of a single wire 14 having one end fixedly interconnected within the tubular member 24, allows production of a needle assembly 20 having an adjustable suture grasping member 10 and having a reduced overall cross-sectional area. That is, as only a single strand of wire 14 is disposed within the hollow bore of the needle assembly 20, a smaller tubular member 24 may be utilized in comparison to a needle assembly that utilizes a collapsible hoop grasping assembly that necessarily requires two wires (i.e., opposing sides of a collapsible hoop) fit within the tubular member 24. Furthermore, when utilizing a single wire grasping assembly, the wire 14 may be tapered providing a wire 14 having a reduced distal end 16 diameter. In this regard, it is possible to produce smaller suture needles assemblies for more delicate surgical applications.

In order to accommodate such sizing reductions while maintaining adequate and strength characteristics, it has been found that the tubular member 24, wire 14 and/or hoop member 52 of the needle assemblies 20 may comprise nickel, titanium, and nickel titanium alloys. It is believed other materials which may be employed for such componentry may include spring steel, stainless steel, as well as composite materials.

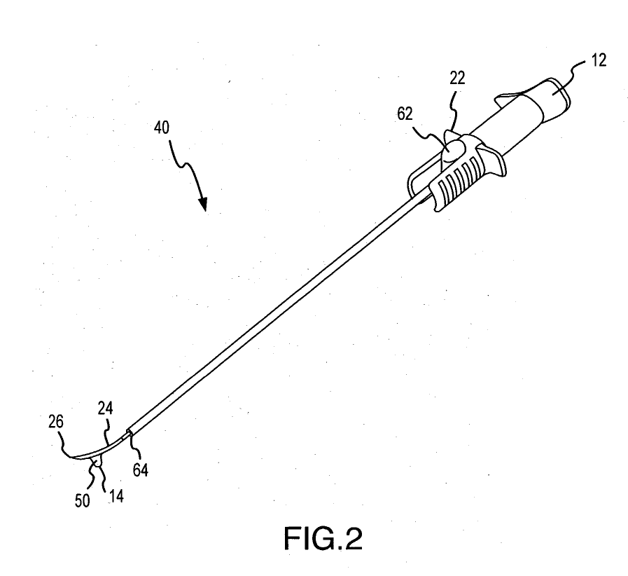

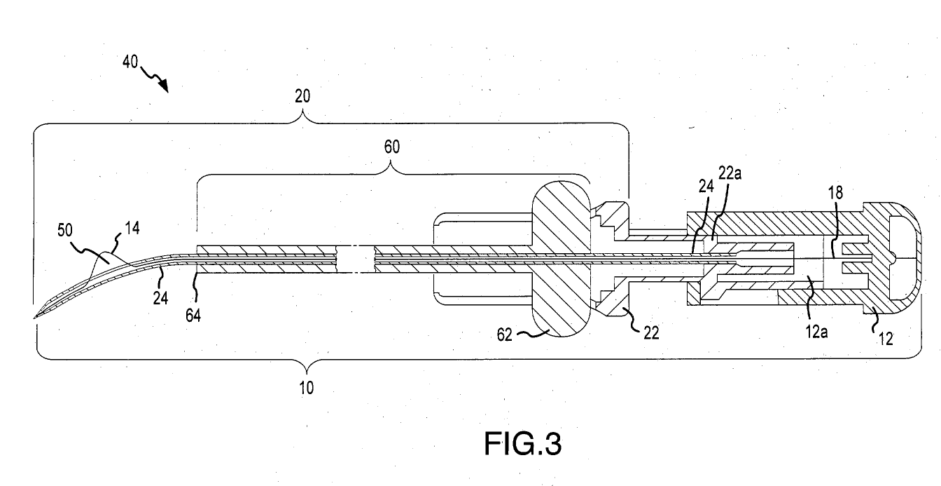

FIGS. 2 and 3 show a perspective and cross-sectional views, respectively, of a laparoscopic device 40 that utilizes the suture needle shown above in the embodiment of FIG. 1A. As shown in the laparoscopic device 40, the needle assembly 20 further includes a proximal needle handle 22 interconnected to the proximal end of the tubular member 24. This proximal needle handle 22 may be utilized for rotating, advancing, and/or retracting the needle assembly 20. Likewise, the grasping assembly 10 (which is shown extended in FIGS. 2 and 3) further includes a proximal grasping handle 12 interconnected to the proximal end 18 of the wire 14 for use in advancing, retracting and/or rotating the wire 14 to selectively deploy and manipulate the loop 50 defined thereby. As will be appreciated, the size of the loop 50 deployed outside the device 40 is controlled by the amount the grasping handle 12 is depressed relative to the needle assembly 20. In addition, suture device 40 includes a pusher tube assembly 60 disposed on the outside surface of the needle assembly 20. The pusher tube assembly 60 includes a pusher handle 62 and a pusher end 64 located proximally to the distal end of the needle assembly 20. The pusher handle 62 is utilized for selectively positioning (i.e., pushing) suture material and/or a pre-tied knot relative to the distal end of the needle assembly 20 during suturing procedures. That is, suture material including a pre-tied knot may be disposed around (i.e., wound) the tubular member 24 immediately in front of the pusher end 64 of the pusher tube assembly 60. In addition, the pusher tube assembly 60, along with the needle assembly 20, may be utilized to selectively receive a suture cartridge including a pre-tied knot to simplify internal suturing procedure. Such a suturing apparatus is illustrated in co-pending U.S. patent application Ser. No. 09/662,936 entitled “Improved Suturing Apparatus and Method” having a filing date of Sep. 15, 2000, the entirety of which is incorporated herein by reference as if set forth in full.

From the inside out cross-sectional view shown in FIG. 3, the grasping assembly 10, needle assembly 20, and pusher tube assembly 60 are all disposed in a concentric coaxial relationship for separate and selective advancement/retraction and/or rotation relative to one another. Furthermore, any or all of these coaxially aligned elements may contain a predetermined frictional interface to permit co-rotation, if desired. As shown, the laparoscopic device 40 is in a deployed configuration where the grasping wire 14 is deployed through the slot opening 28. It will be noted that the wire 14 is a single continuous strand extending from its attachment point near the piercing tip 26 of the needle assembly 20 to the proximal grasping handle 12, where it is fixedly interconnected. As there are no interconnecting or intermediate members, this continuous wire provides increased tensile strength. Likewise, the hollow tubular member 24 of the needle assembly 20 extends over a majority of the length of the suturing device 40 between the piercing tip 26 and the proximal needle handle 22. It will be noted that the needle handle 22 contains an internal shaft portion 22A that is sized for slidable receipt within an opening 12A of the grasping handle 12. Further, it will be appreciated that a spring (not shown) may be inserted between the opening 12a of the grasping handle 12 and the shaft portion 22A of the needle handle 22. In this regard, the spring may provide a biasing force that maintains the grasping assembly 10 in the non-deployed configuration except for when a surgeon purposely advances the grasping handle 12 relative to the needle handle 22. Finally, it will be noted that the pusher handle 62 is designed to slidably fit within a mating needle assembly handle slot (see FIG. 2). As will be appreciated, the pusher tube assembly 60 is a continuous tube and, by advancing the pusher tube handle 62, the distal pusher end 64 may be selectively advanced relative to the distal end of the needle assembly 20.

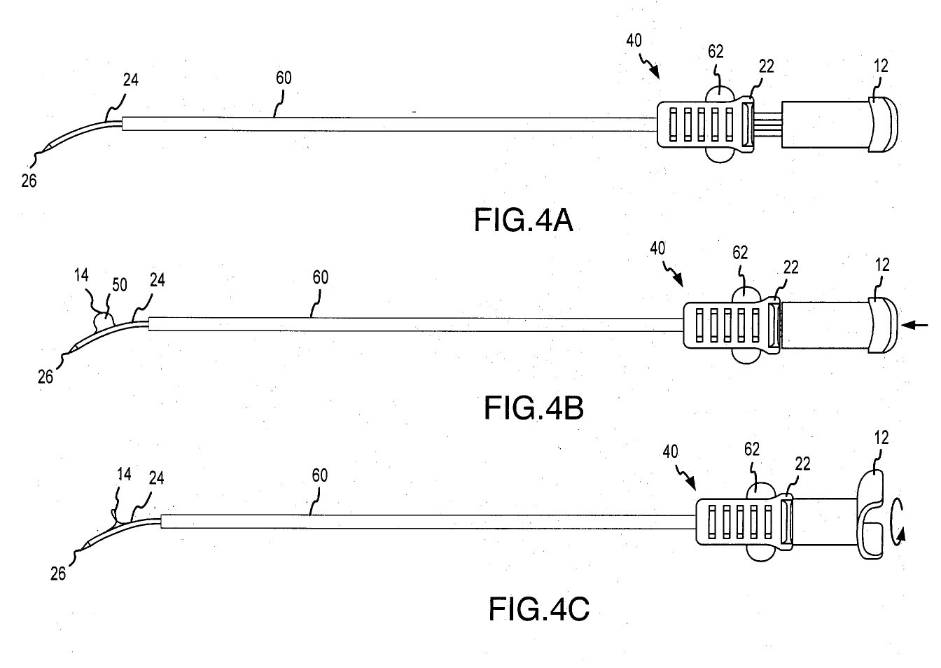

FIGS. 4A, 4B and 4C illustrate the grasping assembly 10 in a non-deployed state, in a deployed state, and in a deployed rotated state, respectively. As shown in FIG. 4A, when the grasping assembly is in the non-deployed state, the proximal grasping handle 12 is extended relative to the proximal needle handle 22. That is, the grasping wire 14 is fully disposed within the hollow tubular member 24 of the needle assembly 20. By advancing the proximal grasping handle 12 relative to the proximal needle handle 22 (see FIG. 4B), the wire 14 is extended out of the slot opening 28 on the distal end of the device 40 to provide a loop 50 relative to the outside surface of the needle assembly 20. As shown in FIG. 4C, while the grasping handle 12 is advanced relative to the needle handle 22, rotating the grasping handle 12 causes the loop 50 defined by the wire 14 to twist. That is, the plane defined by the loop 52 is no longer aligned with the longitudinal axis of the needle assembly 52. When a rotational force is applied to the wire 14, the loop 50 can rotate (i.e. twist) about an axis transverse to the longitudinal axis of the needle assembly 20. This selective twisting of the loop 50 allows for increased flexibility for a surgeon to engage suture material during surgical procedures.

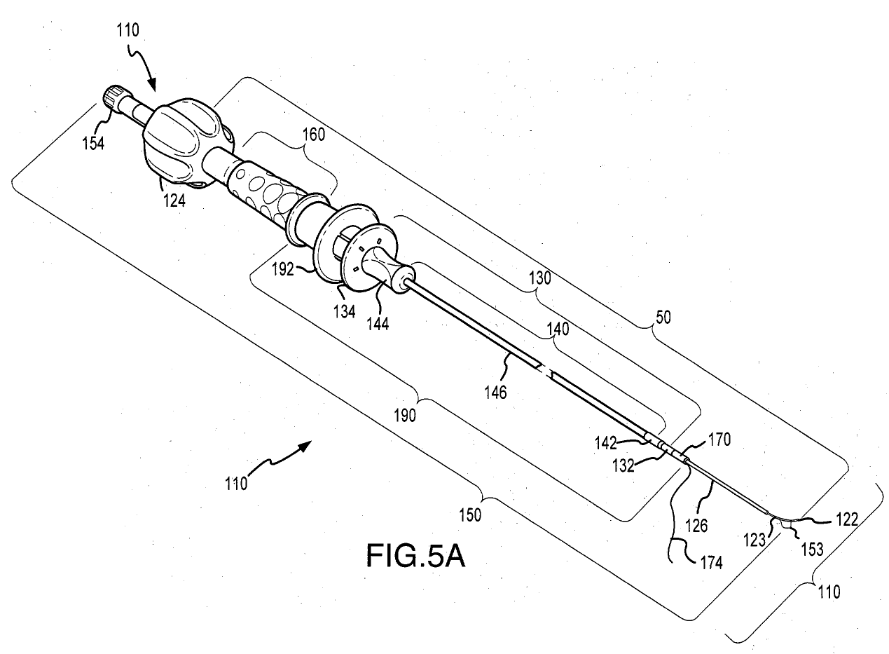

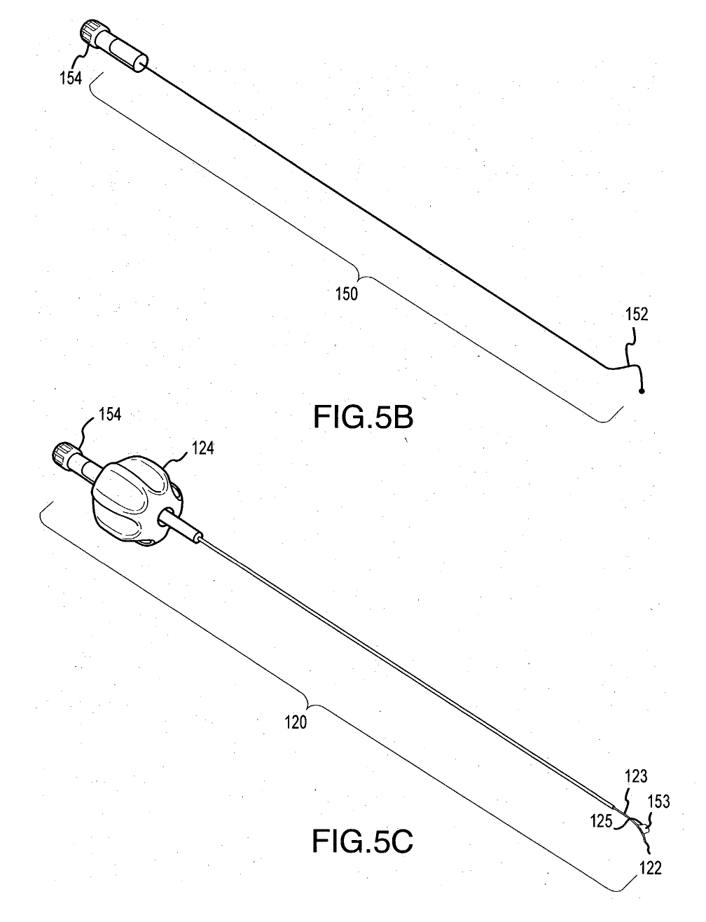

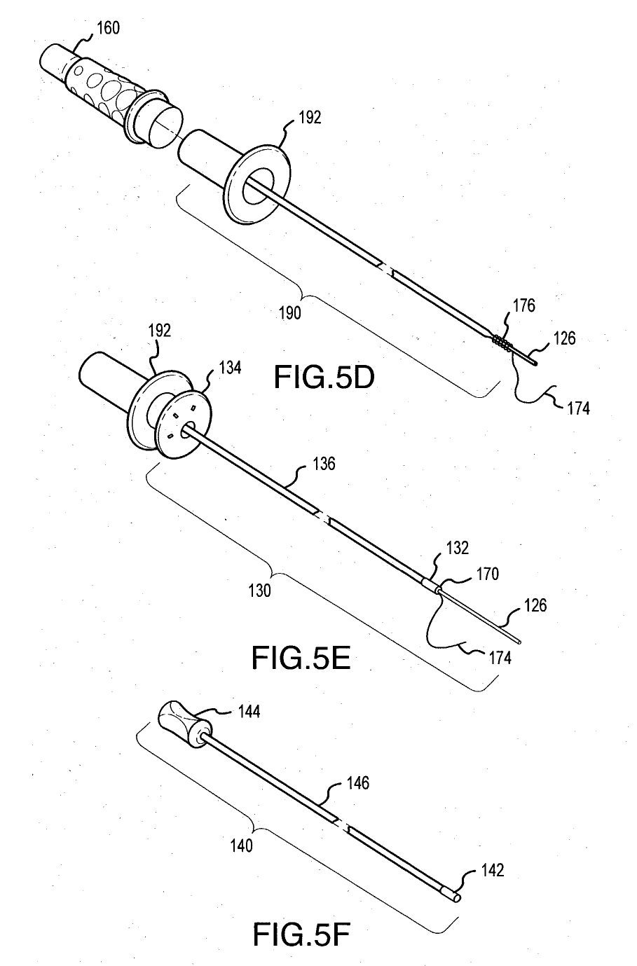

FIGS. 5A-F, 6A-C, and 7A-B illustrate another embodiment of an endoscopic suturing apparatus 110 utilizing the suture needle of FIG. 1A. However, it will be noted that any of the suture needle embodiments as shown in FIGS. 1A-1D may be utilized. The suturing apparatus 110 shown in FIG. 5A comprises an elongated needle assembly 120 having a hollow tubular member 123 terminating in a closed piercing tip 122 at one end and a rotatable and advanceable/retractable handle 124 at a proximal end. The suturing apparatus 110 further includes a spool assembly 190 and a spool 126 about which a length of suturing material with a pre-tied knot may be disposed (not shown in FIG. 5A). In this regard, the suturing apparatus 110 also includes a knot drop assembly 130 having a pusher end 132 positionable adjacent to the spool 126 and further having a selectively advanceable/retractable knot pushing handle 134 at a proximal end.

The spool 126 forms the distal end of the spool assembly 190. The spool assembly 190 further includes an advanceable/retractable handle 192 at a proximal end for selectively advancing and retracting the spool 126 to increase/decrease the tension of suture material disposed between the spool 126 and the piercing tip 122, as will be more fully discussed herein. The suturing apparatus 110 also includes a suture cutter assembly 140 comprising a distal end 142 with a suture cutting surface positionable adjacent to the pusher end 132 of the knot positioning assembly 130 and spool 126 of the slacker assembly 190. The suture cutter assembly 140 further includes an advanceable/retractable handle 144 at a proximal end.

The suture apparatus 110 also includes a suture grasping assembly 150 having a grasping wire 152 for selective advancement out/retraction within a side aperture 125 (not shown in FIG. 5A) of the hollow tubular member 123 near the piercing tip 122 of the needle assembly 120 to define a grasping loop 153. The grasping assembly further includes a selectively advanceable/retractable and rotatable handle 154 provided at a proximal end. Finally, the suturing apparatus 110 includes a proximal primary handle portion 160 for use in manipulating the suturing apparatus 110. The various components of the suturing apparatus 110 are disposed in concentric coaxial relation for separate and selective advancement/retraction and/or rotation relative to one another. For illustrative purposes, FIGS. 5B-5F show one embodiment of each component from an inside-out perspective. In particular, FIG. 5B shows the grasping assembly 150; FIG. 5C shows needle assembly 120 with the grasping assembly 150 coaxially disposed therein; FIG. 5D shows the spool assembly 190 including suture material 174 and a pre-tied knot 176; FIG. 5E shows the knot drop assembly 130; and FIG. 5F shows the suture cutter assembly 140. It will be noted that the grasping assembly 150 and needle assembly 120 may be disposed with a predetermined frictional interface to permit co-rotation if desired. Similarly, the proximal handle 124 of the needle assembly 120 and the primary handle 160 may be disposed with a predetermined frictional interface to facilitate co-rotation if desired.

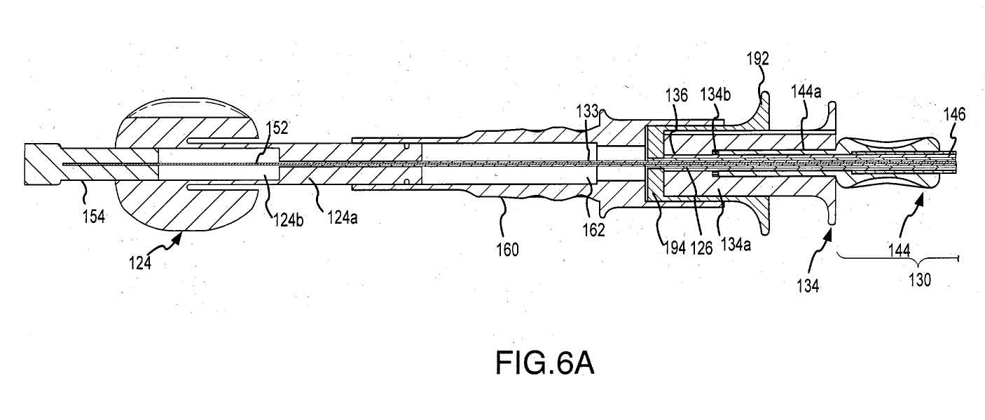

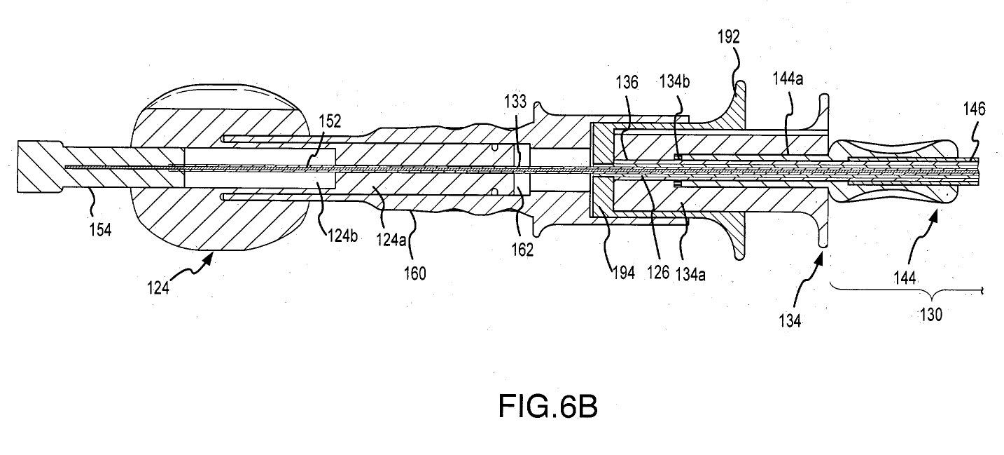

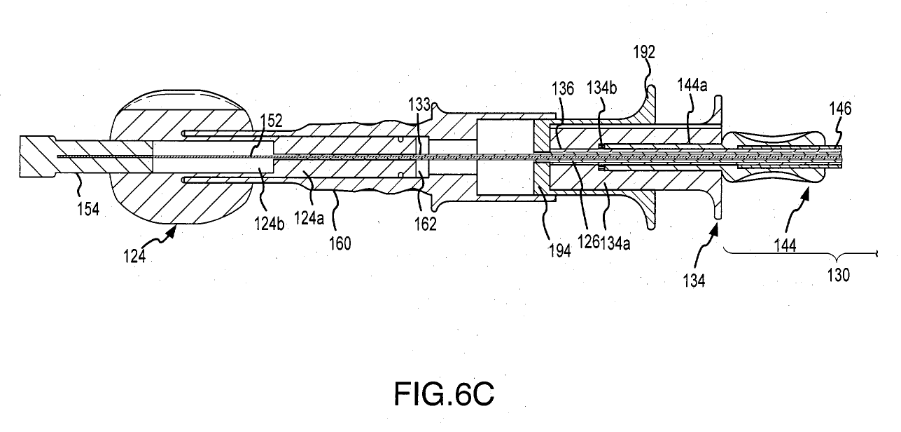

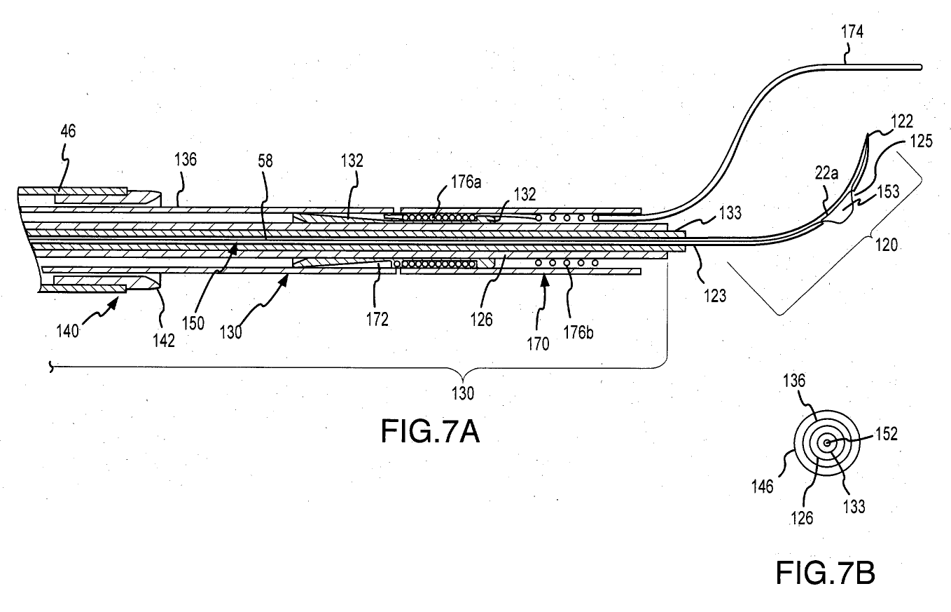

Reference is now made to FIGS. 6A-C and 7A-B, which illustrate distal and proximal cross-sections of the suturing apparatus 110 of FIG. 5A, respectively. With particular respect to grasping assembly 150, FIG. 7A illustrates the grasping wire 152 in an extended, deployed position defining a grasping loop 153 outside the needle assembly 120. When the grasping wire 152 is advanced through the side aperture 125 of the tubular member 123 near the piercing end 122 of the needle assembly 120, the loop 153 is available for selectively grasping a suture 174 in the suturing process. The wire 152 utilized to form the loop 153 of the grasping assembly 150 has a first end interconnected to an inside surface of the hollow tubular member 123 adjacent to the piercing tip 122 and second end affixed to the proximal handle 154 as shown in FIGS. 6A-C. In the latter regard, it can be seen in FIGS. 6A-C, that handle 154 is disposed for rotatable and slideable engagement through the handle 124 of the needle assembly 120. For such purposes, an aperture 124b is formed through the handle 124. With respect to needle assembly 120, FIG. 7A illustrates that the hollow tubular member 123 may be defined by a separate end section and an intermediate driver tube member 133 slideably received within the spool 126. The proximal end of the intermediate driver tube 133 is fixedly connected to an internal shaft portion 124a of the proximal handle 124. This shaft portion 124a is sized for slideable receipt within an opening 162 of the primary handle 160 of the suturing apparatus 110.

It should be noted that the spool 126 of the spool assembly 190 extends the substantial length of suture apparatus 110 for interconnection with the proximal spool handle 192. In this regard, as shown in FIGS. 6A-6C, the driver tube 133 of the needle assembly 120 is slidably received within the spool tube of the spool assembly 190. In turn, the proximal end of the spool tube 126 is interconnected to an internal shaft portion of the proximal spool handle 192.

Referring again now to FIG. 7A, a length of suture material 176 is shown wound about the spool 126 of the needle assembly 120. A first end 172 of the suture material 170 is interconnected to the pusher end 132 of knot positioning assembly 130 while a second free end 174 of the suture material 170 is shown in a position extending away from pusher end 132 of the knot drop assembly 130. A first wound portion 176a is utilized to anchor the suture material to the suture device 110 while a second wound portion 176b of suture material 170 includes a pre-tied knot (e.g., a Roeder knot). It should be noted that in connection with the use of suture apparatus 110 the second end 174 of the suture material 170 may be grasped and retracted through the side aperture 125 of the needle assembly 120 by the grasping assembly loop 153. Further, during use of suture apparatus 110 the pre-tied knot portion 174 may be selectively positioned by use of the knot positioning assembly 130. That is, handle 134 of the knot positioning assembly 130 may be selectively advanced and/or handle 124 of the needle positioning assembly 120 may be selectively retracted so that the pre-tied knot of suture material 170 is “dropped” beyond the distal end of needle assembly 120.

With further respect to the knot drop assembly 130, FIG. 7A illustrates that the distal pusher tip 132 may be defined by a separate piece that fits within the end of an elongated tube member 136 of the pusher assembly 130. This separate piece or pusher tip 132 may be interconnected with the distal end of the tube member 136 in any appropriate manner, including but not limited to, utilizing adhesives as well as a mechanical interference fit. In turn, the elongated tube member 136 extends substantially the entire length of suturing apparatus 110 for interconnection with the proximal handle 134. Further, and as best shown by the concentric relationship diagram of FIG. 7B, the spool tube 126 of the spool assembly 190 may be slideably received within the tube member 136 of the knot drop assembly 130. The proximal end of the tube member 136 is interconnected to an internal shaft portion 134a of the proximal handle 134 which is sized to be slideably received within a cylindrical opening 194 of the slacker handle 192 of the suturing apparatus 110.

As to the suture cutter assembly 140, FIG. 5F illustrates that a separate member may define the distal cutting end 142. In turn, the distal end member 142 may be securely received within the open end of an elongated tube member 146 that extends the substantial length of suturing apparatus 110. As shown in FIGS. 6A-6C, the tube member 136 of the knot drop assembly 130 may be slideably received within the tube member 146 of the suture cutter assembly 140. The proximal end of the tube member 146 may be interconnected to an internal shaft portion 144a of the proximal handle 144. Shaft portion 144a is sized to be slideably received within a cylindrical opening 134b of the proximal handle 134 of the knot positioning assembly 130.

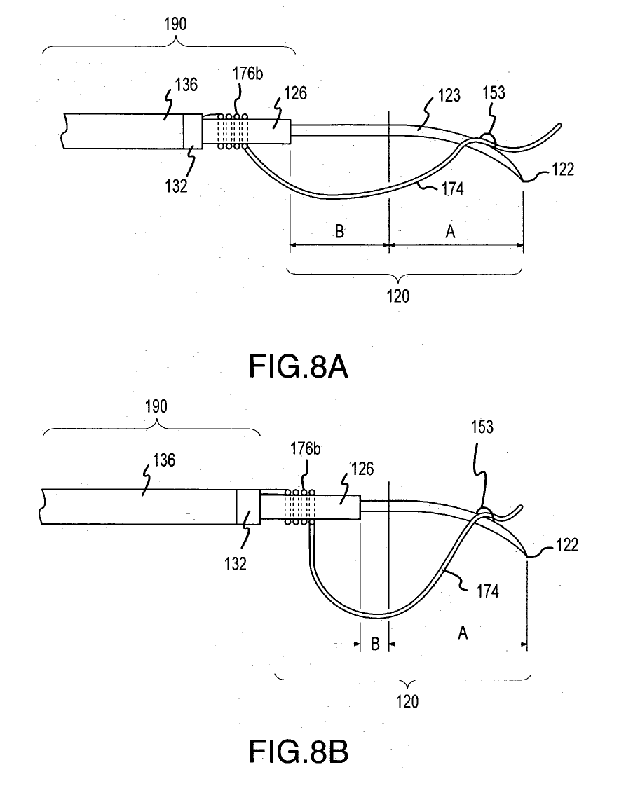

Referring to FIGS. 6A-C, 8A and 8B, utilization of the spool assembly 190 and needle assembly 120 to selectively adjust the slack in the suture material 174 between the second wound portion 176b and the piercing tip 122 is described. As shown in FIG. 6B, the proximal needle handle 124 is in a forward position relative to the primary handle 160 and the spool knob 192 is in a full back position relative to the primary handle 160. In this configuration, the needle assembly 120 is a fully extended position and the suture material 174 is in an extended configuration shown in FIG. 8A. By retracting the proximal needle handle 124 relative to the primary handle, see FIG. 6A, the piercing tip 122 may be moved a distance (a) (to the left as shown) providing slack in the suture material 174. Referring to FIG. 6C, by advancing the spool handle 192 relative to the primary handle 160, the spool 126 may advance towards the piercing tip 122 a distance (b) (to the right as shown) again providing additional slack in the suture material 174. As may be appreciated, through selective advancement retraction of the proximal handles 124, 192 relative to the primary handle 160 the suture material 174 may be in a relatively taut configuration (FIG. 8A) or in a loose configuration (FIG. 8B) or in any desired intermediate configuration to facilitate in suturing procedures.

As noted, the suturing apparatus 110 is particularly adapted for endoscopic applications. In this regard, it may be appreciated that portions of the grasping assembly 150, spool assembly 190, needle assembly 120, knot drop assembly 130 and suture cutter assembly 140 may be of a flexible construction to allow for passage through an endoscope designed for passage through oral, anal, and other endoscopic access locations/canals.

Further, for typical endoscopic applications, the length of driver tube 133, spool 126, tube members 136 and 146, as well as the length of the wire 152 should preferably be at least about 70″, and the outer diameter of tube member 146 of the suture cutter assembly 140 should preferably not exceed about 0.562″. Correspondingly, the outer diameters of tube member 136, spool 126, driver tube 133 and wire 152 should be established in a decreasing, concentric fashion. By way of example, the above noted components may have diameters in ranges in such a manner as to have a clearance that will allow free movement without adverse friction.

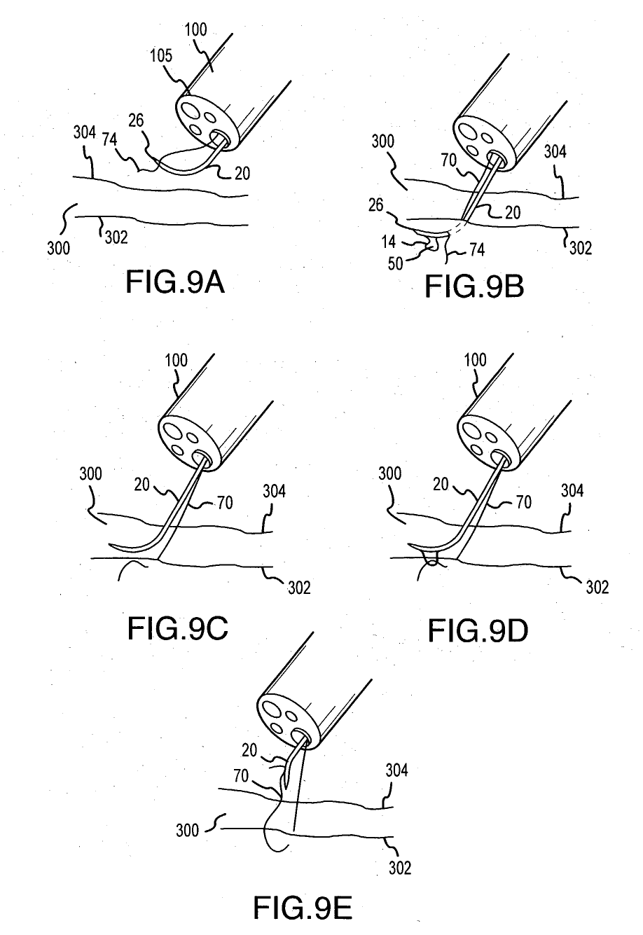

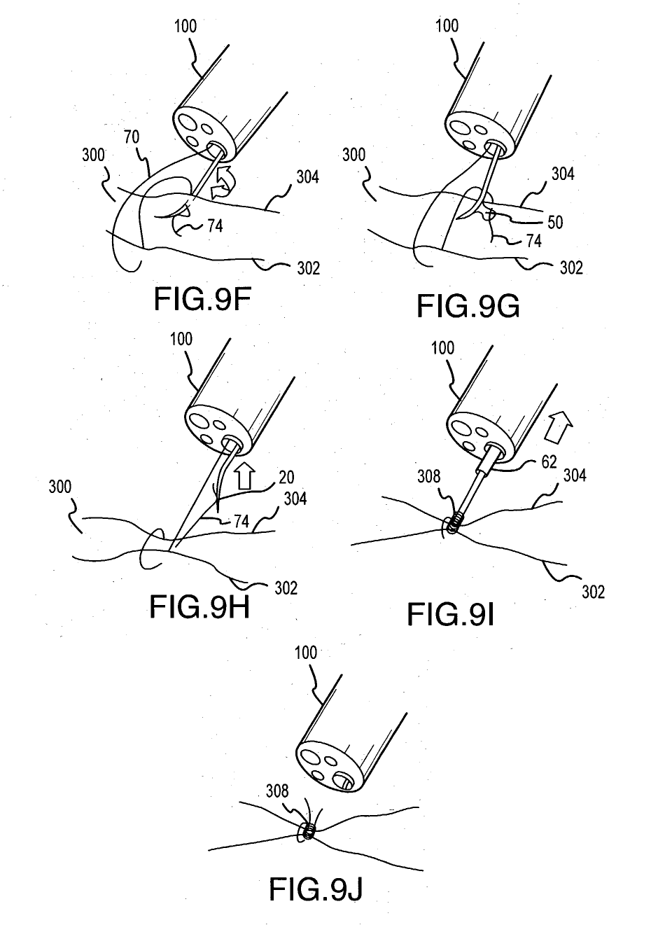

Referring now to FIGS. 9A-9J, an exemplary suturing procedure is described utilizing the suturing needle of the present invention. As will be appreciated, the following procedure may be performed using either, the laparoscopic device 40 of FIG. 2 or the endoscopic suturing device of FIG. 5A. As shown, the needle assembly 20 of FIG. 1A is disposed through a first port of a flexible endoscope device 100. Prior to suturing, the flexible endoscope device 100 is inserted into a patient's body with the distal end 108 positioned adjacent to a tissue region of interest. Additional, medical devices may be disposed through one or more of the remaining ports of the cannula device 100 to facilitate performance of medical procedures to the tissue site of interest.

As illustrated in FIG. 9A, it can be seen that the suturing assembly 20 has been advanced through the flexible endoscope device 100, so that the piercing tip 26 of the needle assembly 20 projects from the distal end 108 of the flexible endoscope device 100 adjacent to a surgical incision 300 that forms first and second tissue portions 302, 304. In the illustrated embodiment, a free end 74 of suture material has been previously disposed between the grasping wire 14 (not shown) of the grasping assembly 10 and the hollow tubular member 24 of the needle assembly 20. Initially, the piercing tip 26 of the needle assembly 20 is advanced to a desired position relative to the incision 300. This may entail by manipulation of the needle assembly handle 22 to advance and/or rotate the piercing tip 26 into contact with the tissue at the incision 300, whereupon, the needle assembly 20 and piercing tip 26 may be advanced through a portion of the tissue 302 as shown in FIG. 9B. Accordingly, the grasped suture material 74 is placed through the first side 302 of the incision 300.

At this point, the grasping handle 12 of the device 40 may be advanced relative to the needle handle 22 to advance the wire 14 through the slot opening 28 so that the loop 50 is disposed outside the needle assembly 20, See FIG. 9B. Once, the needle assembly 20 has released the free end 74 of the suture material 70, see FIG. 9C, the grasping handle 12 may be released relative to the needle handle 22 such that the loop 50 is drawn back into the hollow tubular member 24. The needle assembly 20 may then be withdrawn back out of the first side 302 of the tissue while the suture material 70 remains extended through the tissue, see FIG. 9C. Thereafter, the grasping assembly 10 may be again extended to define the loop 50, as shown in FIG. 9D. Once disposed relative to the outside surface of the needle assembly 20, the grasping loop 50 engages the free end 74 of the suture material 70 that extends through the first side 302 of the incision 300. Again, this manipulation may involve advancement/retraction of the entire needle assembly 20, rotation of the needle assembly 20 and/or rotation of the grasping handle 12 to dispose the free end 74 of the suture material through the loop 50. Once positioned through the loop 50, the loop 50 is withdrawn into the slot opening 26, trapping the suture material 70 between the wire 14 and the hollow tubular member 24 of the needle assembly 20. See FIG. 9E. The suture material 70 may then be engaged through the second side 304 of the incision in a manner substantially similar to the above described processes. See FIGS. 9F and 9G. Once a predetermined number of sutures are made between the first and second sides 302, 304 of the incision 300, the needle assembly 20 may be retracted to cinch the suture (see FIG. 9H), drawing the sides 302, 304 of the incision 300 together. Accordingly, the pusher assembly 62 may be utilized to push a pre-tied knot beyond the piercing tip 26 of the needle assembly (see FIG. 91). Accordingly, this knot is utilized to secure the sutured sides together (see FIG. 9J).

The embodiments described above are for exemplary purposes only and is not intended to limit the scope of the present invention. Various adaptations, modifications and extensions of the embodiment will be apparent to those skilled in the art and are intended to be within the scope of the invention as defined by the claims which follow.

What is claimed:

1. A suturing apparatus, comprising:
a needle terminating at a distal end for engaging patient tissue and having an internal passage extending through at least a portion of said needle to a side aperture thereof, and
a grasping member extending through at least a portion of said internal passageway, wherein at least a portion of said grasping member is selectively moveable through said internal passageway and through said side aperture for grasping a suture.

2. The apparatus as recited in claim 1, wherein said needle is closed at said distal end.

3. The apparatus as recited in claim 1, wherein said side aperture is spaced from said distal end.

4. The apparatus as recited in claim 1, wherein said internal passageway is configured to restrict the movement of at least a first portion of said grasping member.

5. The apparatus as recited in claim 4, wherein a first section of said internal passageway is reduced to restrict the movement of said at least a first portion of said grasping member.

6. The apparatus as recited in claim 1, wherein said grasping member comprises:
a filament extending through at least a portion of said internal passageway, wherein at least a portion of said filament is moveable through said internal passageway upon selective relative movement between said needle and said grasping member.

7. The apparatus as recited in claim 6, wherein a distal end of said filament is fixedly interconnected to said needle near said distal end of said needle.

8. The apparatus of claim 7, wherein said distal end of said filament is fixedly interconnected within said internal passageway of said needle.

9. The apparatus of claim 8, wherein said distal end is fixedly interconnected within said passageway between said side aperture and said distal end of said needle.

10. The apparatus as recited in claim 7, wherein said filament comprises a single continuous strand between said distal end and a proximal end.

11. The apparatus as recited in claim 7, wherein axial movement of said filament relative to said needle results in one of:
advancing a portion of said filament within said internal passageway through said side aperture, wherein said portion of said filament and an outside surface of said needle in combination define a loop for grasping a suture; and
retracting a portion of said filament which, in combination with an outside surface of said needle, define a loop, through said side aperture into said internal passageway.

12. The apparatus as recited in claim 11, wherein said filament comprises a resilient material to permit flexural curvature along a length thereof and wherein, upon said filament defining a loop outside of said needle, rotational movement of said proximal end of said filament causes at least one of:
an arcuate movement of said portion of said filament disposed through said aperture relative to a longitudinal axis of said needle; and
a twisting of said portion of said filament in an axis transverse to a longitudinal axis of said needle.

13. The apparatus as recited in claim 6, wherein said distal end of said filament comprises a preformed loop for selective positioning through said side aperture.

14. The apparatus as recited in claim 13, wherein said loop comprises:
a pre-formed portion, wherein said preformed loop portion collapses to a non-deployed state upon positioning within said internal passageway and automatically opens to a deployed state when positioned through said side aperture.

15. The apparatus as recited in claim 14, wherein said spring-loaded portion is of a hoop-like configuration for selective positioning about an end portion of a surgical suture in said deployed state and for capturing said end portion of said suture in said non-deployed state.

16. The apparatus as recited in claim 1, further comprising:
suture material having a pre-tied knot disposed about a portion of said needle.

17. The apparatus as recited in claim 16, wherein said side aperture is disposed between said suture material and said distal end of said needle.

18. The apparatus as recited in claim 16, further comprising a pusher member disposed to permit selective relative movement with said needle, wherein said pusher is selectively operable to position said pre-tied knot beyond said distal end of said needle.

19. The apparatus of claim 18, wherein said needle and said pusher member are in a concentric coaxial relationship.

20. The apparatus of claim 16, wherein said suture material further comprises first and second ends.

21. The apparatus of claim 20, wherein one of said first and second ends is affixed relative to a proximal portion of said apparatus and the other of said first and second ends is selectively engageable by said grasping member.

22. The apparatus of claim 1, wherein said needle comprises a material selected from a group consisting of: nickel titanium alloy, nickel, titanium, stainless steel and surgical steel.

23. The apparatus of claim 1, wherein said grasping member comprises a material selected from a group consisting of: nickel titanium alloy, nickel, titanium, stainless steel, and surgical steel.

24. A suturing apparatus, comprising:
a needle terminating at a distal end for engaging patient tissue and having an internal passageway extending through at least a portion of said needle; and
a grasping member having first and second ends and extending through at least a portion of said internal passageway, wherein at least a first portion of said grasping member is restricted from passing through said internal passageway;
said needle and said grasping member permitting selective relative movement therebetween and wherein, upon said selective relative movement, a second portion of said grasping member is positionable through an aperture on said needle for grasping a suture.

25. The apparatus as recited in claim 24, wherein said internal passageway is reduced in at least one dimension to restrict passage of said at least one of said first and second ends of said grasping member.

26. The apparatus as recited in claim 24, wherein one of said first and second ends of said grasping member is fixedly interconnected to said needle.

27. The apparatus as recited in claim 26, wherein one of said first and second ends of said grasping member is fixedly interconnected within said internal passageway of said needle.

28. The apparatus as recited in claim 27, wherein one of said first and second ends of said grasping member is fixedly interconnected within said internal passageway between said distal end and said aperture.

29. The apparatus as recited in claim 26, wherein said grasping member comprises a single continuous filament between said first and second ends.

30. The apparatus as recited in claim 24, wherein a relative axial movement between said filament and said needle results in one of:
advancing said second portion of said filament between said first and second ends within said internal passageway through said aperture to at least partially define a loop outside of said needle for grasping a suture; and
retracting said second portion of said filament at least partially defining a loop outside said needle through said aperture into said internal passageway.

31. The apparatus as recited in claim 24, wherein said aperture is formed through said distal end of said needle.

32. The apparatus as recited in claim 24, wherein said aperture is formed through a side surface of said needle.

33. A method for utilizing a suturing apparatus, comprising:
advancing a portion of a grasping member at least partially disposed within an internal passageway of a needle through an aperture extending through a side surface of said needle to at least partially define a loop outside of said needle;
engaging a portion of a suture material through said loop; and
retracting said portion of said grasping member at least partially defining a loop outside said needle through said aperture, wherein said loop secures said suture material relative said needle.

34. The method of claim 33, wherein said steps of advancing and retracting further comprise applying an axial force to a proximal end of said grasping member to produce an axial movement of said grasping member relative to said needle.

35. The method of claim 33, wherein said step of engaging further comprises at least one of:
rotating said needle, wherein said grasping member and said loop at least partially defined by said grasping member rotate with said needle to dispose said portion of said suture material through said loop; and
rotating the distal end of said grasping member while maintaining said needle in a fixed position, wherein said rotating step causes said loop to move relative to a longitudinal axis of said needle and to dispose said portion of said suture material through said loop.

36. The method of claim 35, further comprising:
applying a torsional force to a proximal portion of said grasping member to alter the planar configuration of said loop relative to said needle.

37. The method of claim 33, wherein said retracting step further comprises pulling at least a portion of said suture material disposed through said loop into said aperture of said internal passageway of said needle.

38. The method of claim 37, wherein said step of advancing comprises advancing a preformed loop member associated with said grasping member through said aperture.

39. The method of claim 33, further comprising:
advancing a portion of said needle and said suture material clasped between said grasping member and said needle through a patient tissue site, wherein said suture material is pulled through said patient tissue site by said needle;
releasing said portion of said suture material; and
withdrawing said portion of said needle from said patient tissue site, wherein said portion of said suture material is left protruding from said patient tissue site.

40. The method of claim 39, wherein said step of releasing further comprises:
applying an axial force to the proximal end of said grasping member to dispose a portion of said grasping member through said aperture and define said loop outside of said needle;
disengaging said portion of said suture material through said loop; and
retracting said grasping member through said aperture into said internal passageway.

41. The method of claim 39, wherein said steps of engaging, retracting, advancing, releasing and withdrawing are performed a plurality of times to form a plurality of sutures through said patient tissue site.

42. The method of claim 39, wherein a pre-tied knot portion is initially positioned about said suture needle and wherein said method further comprises:
engaging said portion of said suture material protruding from said patient tissue site; and
pushing said pre-tied knot portion off of said needle, wherein said pre-tied knot secures said suture material disposed through said patient tissue site.

43. The method of claim 42, wherein pushing further comprises:
pushing said pre-tied knot over said portion of suture material, wherein said portion of suture material is disposed within said pre-tied knot.
